(19) United States
(12) Patent Application Publication
(10) Pub. No.: US 2016/0010119 A1
(43) Pub. Date: Jan. 14, 2016

(54) PROCESSES FOR THE ACIDIC, ANAEROBIC CONVERSION OF HYDROGEN AND CARBON OXIDES TO OXYGENATED ORGANIC COMPOUND

(71) Applicant: Coskata, Inc., WARRENVILLE, IL (US)

(72) Inventor: Robert Hickey, OKEMOS, MI (US)

(21) Appl. No.: 14/327,249

(22) Filed: Jul. 9, 2014

(51) Int. Cl. C12P 7/16; C12P 7/04; C12P 7/06

(52) U.S. Cl. CPC C12P 7/16; C12P 7/06; C12P 7/04

(57) ABSTRACT

Processes for the bioconversion of syngas to oxygenated organic compound are disclosed that reliably, cost-effectively and efficiently supply sulfur nutrient to microorganisms contained in acidic, aqueous fermentation menstrua. In the processes of this invention, at least a portion of the sulfur nutrient for the population of microorganisms in the aqueous menstruum is provided as calcium sulfite, and the presence of undissolved calcium sulfite is maintained in the aqueous menstruum.

### FIELD OF THE INVENTION

This invention pertains to processes for the anaerobic conversion of hydrogen and carbon oxides to oxygenated organic compound wherein undissolved calcium sulfite is maintained in an acidic, aqueous menstruum used for the anaerobic conversion to provide sulfur-containing nutrient to microorganisms used for the anaerobic conversion.

### BACKGROUND

Anaerobic fermentations of hydrogen and carbon monoxide involve the contact of a gaseous substrate-containing feed with an aqueous fermentation menstruum containing microorganisms capable of generating oxygenated organic compounds such as ethanol, acetic acid, propanol and n-butanol. The bioconversion of carbon monoxide results in the production of oxygenated organic compound and carbon dioxide. The conversion of hydrogen involves the consumption of hydrogen and carbon dioxide, and this conversion is sometimes referred to as the H₂/CO₂ conversion or, as used herein, the hydrogen conversion.

Sulfur is a key nutritional need of anaerobic microorganisms used in these fermentations to produce oxygenated organic compound. Any interruptions in the supply of sulfur nutrient results in an almost immediate decrease in the rate and stability of the fermentation.

Organic sulfur sources, such as cysteine, have been used to provide the nutritional sulfur. These organic sulfur sources are expensive, and alternative sources of sulfur to meet this nutritional need have been sought. Less expensive sources of sulfur include, but are not limited to, hydrogen sulfide, and sulfite, bisulfite, thiosulfate and metabisulfite anions. However, typical aqueous menstrua for the bioconversion of carbon monoxide and of hydrogen and carbon dioxide are acidic. Consequently the equilibrium for hydrogen sulfide, which provides the sulfhydryl anion that is believed to be used by the microorganisms, strongly favors gaseous hydrogen sulfide as opposed to the sulfhydryl anion, and gaseous hydrogen sulfide rapidly exits the aqueous menstruum. Sulfite, bisulfite, thiosulfate and metabisulfite are rapidly transformed to sulfide leaving virtually no residual sulfite, bisulfite, thiosulfate or metabisulfite anion in the aqueous menstruum. The sulfide, which will primarily exist as hydrogen sulfide, thus will rapidly evolve from the aqueous menstruum into the gas phase.

Even dosing the aqueous menstruum with sulfite, bisulfite, thiosulfate or metabisulfite anion at levels well in excess of the cell sulfur requirements may not provide the needed sulfur nutrient required to maintain the population of microorganisms in the event of a disruption in their supply. And such overdosing results in the tail gas from the anaerobic bioconversion process having significant concentrations of hydrogen sulfide. Thus, in addition to increasing the cost of supplying sulfur nutrient, accommodations may be required to remove or reduce the concentration of sulfur compounds in the tail gas to enable the use or disposal of the tail gas.

Accordingly, processes for the anaerobic conversion of carbon monoxide and of hydrogen and carbon dioxide to oxygenated organic compounds are sought that can reliably, cost-effectively and efficiently supply sulfur nutrient to the microorganisms under acidic bioconversion conditions without undue concentrations of sulfur compounds being present in the tail gas from a fermentation.

### SUMMARY

By this invention, processes for the bioconversion of syngas to oxygenated organic compound are provided that reliably, cost-effectively and efficiently supply sulfur nutrient to microorganisms contained in acidic, aqueous fermentation menstrua. In preferred embodiments, the supply of sulfur nutrient occurs without undue concentrations of hydrogen sulfide being contained in off-gases from the aqueous menstruum. In accordance with the processes of this invention, at least a portion of the sulfur nutrient for the population of microorganisms in the aqueous menstruum is supplied as calcium sulfite, and the presence of undissolved calcium sulfite is maintained in the aqueous menstruum and, in essence, serves as a reservoir for sulfur nutrient supply.

The presence of undissolved calcium sulfite assures a continuing supply of a low concentration of sulfite anion to the aqueous menstruum. Without wishing to be limited to theory, it is believed that the solubility equilibria of calcium sulfite is such that so long as undissolved calcium sulfite remains in the aqueous menstruum, a dissolved calcium sulfite concentration of between about 20 and 50 milligrams per liter can be maintained in the aqueous menstruum. At these low concentrations, the microorganisms are still able to obtain the needed sulfur nutrient while not generating undue amounts of hydrogen sulfide.

In one broad aspect of the continuous processes of this invention for the anaerobic bioconversion of a gas substrate comprising carbon monoxide, hydrogen and carbon dioxide in an aqueous menstruum containing microorganisms suitable for converting said substrate to oxygenated organic compound, the processes comprise: continuously contacting said gas substrate with said aqueous menstruum under acidic, anaerobic fermentation conditions to bioconvert gas substrate to oxygenated organic compound and provide an oxygenated organic compound-containing menstruum and a depleted gas phase; continuously withdrawing the depleted gas phase from said aqueous menstruum; continuously or intermittently withdrawing a portion of said menstruum for recovery of said oxygenated organic compound, said withdrawal being sufficient to maintain the oxygenated organic compound in said menstruum below a concentration that unduly adversely affects the microorganisms, wherein during the contacting the aqueous menstruum contains undissolved calcium sulfite.

In another broad aspect of the continuous processes of this invention for the anaerobic bioconversion of a gas substrate comprising carbon monoxide, hydrogen and carbon dioxide in an aqueous menstruum containing microorganisms suitable for converting said substrate to oxygenated organic compound, the processes comprise: continuously contacting said gas substrate with said aqueous menstruum under acidic anaerobic fermentation conditions to bioconvert gas substrate to oxygenated organic compound and provide an oxygenated organic compound-containing menstruum and a depleted gas phase; continuously withdrawing the depleted gas phase from said aqueous menstruum; continuously or intermittently withdrawing a portion of said menstruum for recovery of said oxygenated organic compound, said withdrawal being sufficient to maintain the oxygenated organic compound in said menstruum below a concentration that unduly adversely affects the microorganisms, wherein during the contacting undissolved calcium sulfite is provided to the aqueous menstruum in an amount sufficient to maintain undissolved calcium sulfite in the aqueous menstruum. Preferably, the undissolved calcium sulfite is suspended in the aqueous menstruum and is in substantial uniformity in the liquid phase.

In many instances, the pH of the acidic, aqueous menstruum is less than about 6.5, say, between about 4 and 6.0, and most frequently between about 4.5 and 5.5 as lower pH environments favor solventogenesis. Despite the acidity, the depleted gas phase often contains less than about 100, and most frequently less than about 50, parts per million by volume (ppmv) of hydrogen sulfide where calcium sulfite provides substantially all of the sulfur nutrient to the aqueous menstruum.

The calcium sulfite may provide all or a portion of the sulfur requirements for the microorganisms. In many instances, the calcium sulfite, other than indigenous sulfur compounds inherently contained in the feed gas supplying the gas substrate, provides at least about 50 percent, more preferably at least about 80 percent to substantially all, of the sulfur nutrient requirements. The calcium sulfite can also be used as a reservoir of sulfur nutrient source in the event that a disruption in the supply of other sulfur nutrient to the aqueous menstruum occurs. The calcium sulfite can be provided to the aqueous menstruum as calcium sulfite, e.g., as solids or as solids slurried in a suitable liquid, preferably an aqueous liquid, which is not deleterious to the microorganisms. Alternatively or in addition, a soluble salt of sulfite, e.g., an alkali metal salt, including but not limited to, sodium and potassium, and ammonium salt, can be used and precipitated with calcium cation in the aqueous menstruum (in situ precipitate). In some preferred embodiments of the processes of this invention at least 70, and most preferably essentially all, the calcium sulfite is added to the aqueous menstruum in the form of an aqueous slurry.

Usually the calcium sulfite solids in the aqueous menstruum are in the form of finely divided particles, e.g., having a major dimension of up to about 100 microns. These small particulates provide a high surface area per unit of mass to facilitate maintaining the equilibrium between undissolved calcium sulfite and dissolved calcium sulfite. In some instances, the undissolved calcium sulfite may be colloidal particles. Preferably essentially all the undissolved calcium sulfite is of sufficiently small particle size that it can be maintained in a relatively uniform dispersion in the aqueous menstruum. Consequently, where solid calcium sulfite is added to the aqueous menstruum, preferably at least about 50 mass percent of the solids have a maximum particle size dimension of between about 1 and 100 microns.

The calcium sulfite or precursors for the precipitation of calcium sulfite can be added continuously or intermittently to the aqueous menstruum. So long as undissolved calcium sulfite is present, as the concentration of dissolved calcium sulfite anion below the saturation concentration at the conditions of the aqueous menstruum, a driving force exists to cause solubilization of more calcium sulfite. Accordingly, the concentration of undissolved calcium sulfite in the aqueous menstruum is not critical and can vary over a wide range. One advantage of the processes of this invention is that the concentration of undissolved calcium sulfite need not be monitored to match the rate of metabolism of the sulfite anion. Often, the concentration of undissolved calcium sulfite is between about 50 milligrams per liter to about 1 gram or more per liter.

### DETAILED DISCUSSION

All patents, published patent applications, unpublished patent applications and articles referenced herein are hereby incorporated by reference in their entirety.

Definitions

As used herein, the following terms have the meanings set forth below unless otherwise stated or clear from the context of their use.

The use of the terms “a” and “an” is intended to include one or more of the element described.

Oxygenated organic compound means one or more organic compounds containing two to six carbon atoms selected from the group of aliphatic carboxylic acids and salts, alkanols and alkoxide salts, and aldehydes. Often oxygenated organic compound is a mixture of organic compounds produced by the microorganisms contained in the aqueous menstruum.

A bioreactor assembly is an assembly of one or more vessels suitable to contain aqueous menstruum and microorganisms for the bioconversion and can contain associated equipment such as injectors, recycle loops, agitators, and the like.

Biomass means biological material living or recently living plants and animals and contains at least hydrogen, oxygen and carbon. Biomass typically also contains nitrogen, phosphorus, sulfur, sodium and potassium. The chemical composition of biomass can vary from source to source and even within a source. Sources of biomass include, but are not limited to, harvested plants such as wood, grass clippings and yard waste, switchgrass, corn (including corn stover), hemp, sorghum, sugarcane (including bagas), and the like; and waste such as garbage and municipal waste. Biomass does not include fossil fuels such as coal, natural gas, and petroleum.

The abbreviation ppm means parts per million. Unless otherwise stated or clear from the context, ppm is on a mass basis (ppm (mass)) for solids in a liquid medium.

Fossil carbonaceous materials, or fossil fuels, include, but are not limited to, natural gas; petroleum including carbonaceous streams from the refining or other processing of petroleum including, but not limited to, petroleum coke; and lignite and coal.

Aqueous menstruum, or aqueous fermentation menstruum, means a liquid water phase which may contain dissolved compounds including, but not limited to hydrogen, carbon monoxide, and carbon dioxide.

Intermittently means from time to time and may be at regular or irregular time intervals.

A concentration of the oxygenated organic compound below that which unduly adversely affects the rate of growth of the culture of microorganisms will depend upon the type of microorganism and the oxygenated organic compound. An unduly adverse effect on the growth rate means that a significant, usually at least a 20 percent, decrease in the growth rate of the microorganisms is observed in comparison to the growth rate observed in an aqueous menstruum having about 10 grams per liter oxygenated organic compound therein, all other parameters being substantially the same.

Substantial uniformity of a component in liquid phase means that the concentration of that component in the liquid phase is substantially the same throughout a bioreactor. Usually the concentration of the component is within about 0.2 mole percentage points in a uniform liquid phase.

Deep tank bioreactor is a bioreactor having a depth of at least about 10 meters and can be operated to provide a substantial non-uniform substrate composition over the depth of the aqueous menstruum contained in the bioreactor. The term bubble column bioreactor as used herein refers to a deep tank bubble column bioreactor unless otherwise explicitly stated and include deep tank reactors where the gas is introduced as small bubbles to promote mixing. A commercial scale bioreactor has a capacity for aqueous menstruum of at least 1 million, and more preferably at least about 5, say, about 5 to 25 million, liters.

Substrate is one or more of (i) carbon monoxide and (ii) carbon dioxide and hydrogen. A feed gas contains substrate and may contain other components including, but not limited to, recycled off-gas or a fraction thereof and other additives, inerts such as methane and nitrogen, and other components that can be contained in a syngas.

Syngas means a gas, regardless of source, containing at least one of hydrogen and carbon monoxide and may, and usually does, contain carbon dioxide.

Overview

The processes of this invention provide reliable, cost-effective and efficient supply of sulfur nutrient to acid, aqueous fermentation menstrua for the bioconversion of syngas to oxygenated organic compound.

Calcium Sulfite

The processes of this invention use calcium sulfite to provide at least a portion of the sulfur needs for the microorganisms used for the bioconversion of syngas. Calcium sulfite is recognized as a food additive, particularly as a food preservative, and as an antimicrobial agent. The low solubility of calcium sulfate in combination with the sulfur requirement need of the microorganisms enables an effective supply of sulfur nutrient as all or a portion of the sulfur nutrient requirement under normal operation or during an interruption in the supply of a different sulfur nutrient normally used in the syngas bioconversion process. As stated above, the concentration of dissolved calcium sulfite in the aqueous menstruum is often in the range of about 20 to 50 milligrams per liter.

In accordance with the processes of this invention solid calcium sulfite is maintained in the aqueous menstruum. The calcium sulfite can be provided (direct addition or in situ) continuously or intermittently to the aqueous menstruum in amounts sufficient to provide undissolved solids of calcium sulfite. The continuous or intermittent addition may occur substantially over the period of time that the bioconversion process is operating either to provide substantially all or a portion of the sulfur nutrient or as a reservoir in the event of a disruption of the supply of a different sulfur nutrient normally used. Alternatively, calcium sulfite can be used only in the event of a disruption in the feed of a normal sulfur nutrient source, the provision of calcium sulfite may commence when the disruption occurs.

The rate of the provision of calcium sulfite can be determined by any suitable parameters. Analytical techniques exist for a determination of the concentration of calcium sulfite solids in the aqueous menstruum; however, they tend to be time consuming. For instance, an aliquot fraction of the aqueous menstruum can be filtered using a 0.2 micron filter to collect solids and the presence of solid calcium sulfite ascertained by X-ray crystal diffraction. Accordingly and most conveniently, the rate of provision of calcium sulfite is based upon the expected metabolic rate of consumption of sulfur by the population of microorganisms in the aqueous menstruum. Also the sulfite concentration in the aqueous menstruum can be monitored and if it is below saturation, the rate of provision of calcium sulfite is increased. In an indirect method, the hydrogen sulfide concentration in the off-gas from the aqueous menstruum can be monitored. If the concentration changes without change in the population density of the microorganisms, the rate of provision of calcium sulfite can be altered to provide a hydrogen sulfide concentration within a targeted range based on the metabolic activity of the microorganism.

Substrate and Feed Gas

Anaerobic fermentation to produce oxygenated organic compound uses a substrate comprising at least one of (i) carbon monoxide and (ii) carbon dioxide and hydrogen, the latter being for the hydrogen conversion pathway. The feed gas will typically contain nitrogen and methane in addition to carbon monoxide and hydrogen. Syngas can be made from many carbonaceous feedstocks. These include sources of hydrocarbons such as natural gas, biogas, biomass, especially woody biomass, gas generated by reforming hydrocarbon-containing materials, peat, petroleum coke, coal, waste material such as debris from construction and demolition, municipal solid waste, and landfill gas.

Syngas is typically produced by a gasifier, reformer (steam, autothermal or partial oxidation). Any of the aforementioned biomass sources are suitable for producing syngas. The syngas produced thereby will typically contain from 10 to 60 mole % CO, from 10 to 25 mole % CO₂ and from 10 to 75, often at least about 30, and preferably between about 35 and 65, mole % H₂. The syngas may also contain N₂ and CH₄ as well as trace components such as H₂S and COS, NH₃ and HCN. Other sources of the gas substrate include gases generated during petroleum and petrochemical processing and from industrial processes. These gases may have substantially different compositions than typical syngas, and may be essentially pure hydrogen or essentially pure carbon monoxide. The gas substrate may be obtained directly from gasification or from petroleum and petrochemical processing or industrial processes or may be obtained by blending two or more streams. Also, the gas substrate may be treated to remove or alter the composition including, but not limited to, removing components by chemical or physical sorption, membrane separation, and selective reaction.

Oxygenated Compounds and Microorganisms

The oxygenated organic compounds produced by the processes of this invention will depend upon the microorganism or combination of microorganisms used for the fermentation and the conditions of the fermentation. Bioconversions of CO and H₂/CO₂ to acetic acid, n-butanol, butyric acid, ethanol and other products are well known. For example, a concise description of biochemical pathways and energetics of such bioconversions have been summarized by Das, A. and L. G. Ljungdahl, Electron Transport System in Acetogens and by Drake, H. L. and K. Kusel, Diverse Physiologic Potential of Acetogens, appearing respectively as Chapters 14 and 13 of Biochemistry and Physiology of Anaerobic Bacteria, L. G. Ljungdahl eds., Springer (2003). Any suitable microorganisms that have the ability to convert the syngas components: CO, H₂, CO₂ individually or in combination with each other or with other components that are typically present in syngas may be utilized. Suitable microorganisms and/or growth conditions may include those disclosed in U.S. Published Patent Application 20070275447, entitled “Indirect Or Direct Fermentation of Biomass to Fuel Alcohol,” which discloses a biologically pure culture of the microorganism Clostridium carboxidivorans having all of the identifying characteristics of ATCC no. BAA-624; U.S. Pat. No. 7,704,723 entitled “Isolation and Characterization of Novel Clostridial Species,” which discloses a biologically pure culture of the microorganism Clostridium ragsdalei having all of the identifying characteristics of ATCC No. BAA-622; both of which are incorporated herein by reference in their entirety. Clostridium carboxidivorans may be used, for example, to ferment syngas to ethanol and/or n-butanol. Clostridium ragsdalei may be used, for example, to ferment syngas to ethanol.

Suitable microorganisms and growth conditions include the anaerobic bacteria Butyribacterium methylotrophicum, having the identifying characteristics of ATCC 33266 which can be adapted to CO and used and this will enable the production of n-butanol as well as butyric acid as taught in the references: “Evidence for Production of n-Butanol from Carbon Monoxide by Butyribacterium methylotrophicum,” Journal of Fermentation and Bioengineering, vol. 72, 1991, p. 58-60; “Production of butanol and ethanol from synthesis gas via fermentation,” FUEL, vol. 70, May 1991, p. 615-619. Other suitable microorganisms include: Clostridium Ljungdahlii, with strains having the identifying characteristics of ATCC 49587 (U.S. Pat. No. 5,173,429) and ATCC 55988 and 55989 (U.S. Pat. No. 6,136,577) that will enable the production of ethanol as well as acetic acid; Clostridium autoethanogemum sp. nov., an anaerobic bacterium that produces ethanol from carbon monoxide. Jamal Abrini, Henry Naveau, Edomond-Jacques Nyns, Arch Microbiol., 1994, 345-351; Archives of Microbiology 1994, 161: 345-351; and Clostridium Coskatii having the identifying characteristics of ATCC No. PTA-10522 described in U.S. Pat. No. 8,143,037.

Mixed cultures of anaerobic microorganisms can also be used for the bioconversions of syngas to product oxygenated organic compounds. See, for instance, U.S. patent applications Ser. Nos. 13/802,916, filed Mar. 14, 2013, entitled Method For Production Of N-Propanol And Other C3-Carbon Containing Products From Syngas By Symbiotic Arrangement Of C1-Fixing And C3-Producing Anaerobic Microorganism Cultures (Toby, et al.); 13/802,930, filed Mar. 14, 2013, entitled Method For Production Of N-Propanol And/Or Ethanol By Fermentation Of Multiple Substrates In A Symbiotic Manner (Enzein, et al.); 13/802,924, filed Mar. 14, 2013, entitled Method For Production Of N-Propanol And Other C3-Containing Products From Syngas Using Membrane Supported Bioreactor (Datta, et al.) and 13/802,905, filed Mar. 14, 2013, entitled Method For Production Of N-Propanol And Other C3-Containing Products From Syngas By Symbiotic Co-Cultures Of Anaerobic Microorganisms (Datta, et al.). C1-fixing microorganisms include, without limitation, homoacetogens such as Clostridium ljungdahlii, Clostridium autoethanogenum, Clostridium ragsdalei, and Clostridium coskatii. Additional C1-fixing microorganisms include Alkalibaculum bacchi, Clostridium thermoaceticum, and Clostridium aceticum. Symbiotic C3-producing microorganisms capable of growing on ethanol and/or acetate as their primary carbon source include, but are not limited to, Pelobacter propionicus, Clostridium neopropionicum, Clostridium propionicum, Desulfobulbus propionicus, Syntrophobacter wolinii, Syntrophobacter pfennigii, Syntrophobacter fumaroxidans, Syntrophobacter sulfatireducens, Smithella propionica, Desulfotomaculum thermobenzoicum subspecies thermosymbioticum, Pelotomaculum thermopropionicum, and Pelotomaculum schinkii. Pathways for the production of oxygenated organic compounds having three carbons include, but are not limited to, Propionibacterium species (Propionibacterium acidipropionici, Propionibacterium acnes, Propionibacterium cyclohexanicum, Propionibacterium freudenreichii, Propionibacterium freudenreichii shermnanii, Propionibacterium pentosaecum) and several other anaerobic bacteria such as Desulfobulbus propionicus, Pectinatus frisingensis, Pelobacter propionicus, Veillonella, Selenomonas, Fusobacterium, Bacteroides fragile, Prevotella ruminicola, Megasphaera elsdenii, Bacteroides vulgates, and Clostridium, in particular Clostridium propionicum.

Fermentation Broth and Fermentation Conditions

The aqueous fermentation broth will comprise an aqueous suspension of microorganisms and various media supplements. Suitable microorganisms generally live and grow under anaerobic conditions, meaning that dissolved oxygen is essentially absent from the fermentation broth. The various adjuvants to the aqueous fermentation broth may comprise buffering agents, trace metals, vitamins, salts etc. Adjustments in the fermentation broth may induce different conditions at different times such as growth and non-growth conditions which will affect the productivity of the microorganisms. U.S. Pat. No. 7,704,723 discloses the conditions and contents of suitable aqueous fermentation broth for bioconversion CO and H₂/CO₂ using anaerobic microorganisms.

The aqueous menstruum is maintained under anaerobic fermentation conditions including a suitable temperature, say, between 25° C. and 60° C., frequently in the range of about 30° to 40° C. The conditions of fermentation, including the density of microorganisms and aqueous fermentation menstruum composition are preferably sufficient to achieve the sought conversion efficiency of hydrogen and carbon monoxide. The pH of the aqueous menstruum is acidic, often less than about 6.5, say, between about 4 and 6.0, and most frequently between about 4.5 and 5.5.

The rate of supply of the feed gas under steady state conditions to a fermentation bioreactor is preferably such that the rate of transfer of carbon monoxide and hydrogen to the liquid phase matches the rate that carbon monoxide and hydrogen are bioconverted. The rate at which carbon monoxide and hydrogen can be consumed will be affected by the nature of the microorganism, the concentration of the microorganism in the aqueous fermentation broth and the fermentation conditions. As the rate of transfer of carbon monoxide and hydrogen to the aqueous fermentation broth is a parameter for operation, conditions affecting the rate of transfer such as interfacial surface area between the gas and liquid phases and driving forces are important. Preferably the feed gas is introduced into the bioreactor in the form of microbubbles. Often the microbubbles have diameters in the range of 0.01 to 0.5, preferably 0.02 to 0.3 millimeter.

Bioreactors and Assemblies

The bioreactor assembly may comprise one or more bioreactors which may be, with respect to gas flow, in parallel or in series flow. Each bioreactor may be of any suitable design; however, preferably the design and operation provides for a high conversion of carbon monoxide and hydrogen to oxygenated organic compound. Fermentation reactors include, but are not limited to, bubble column reactors; jet loop reactors; stirred tank reactors; trickle bed reactors; biofilm reactors; and static mixer reactors including, but not limited to, pipe reactors. Because of economy of capital cost and operation, deep tank bioreactors are preferred. Regardless of the type of deep tank bioreactor, especially where using microbubbles that promote a stable dispersion of bubbles in the aqueous broth, mixing currents exist that not only assure the relatively uniform aqueous phase composition but also increase the contact time between the gas bubbles and the aqueous broth.

Off-gas (Substrate Depleted Gas) Phase

The substrate depleted gas phase egressing from the aqueous fermentation broth will contain a small fraction of the hydrogen and carbon oxides introduced into the bioreactor assembly as the feed gas. Inerts such as nitrogen and primarily methane will comprise a portion of the depleted gas phase where syngas from steam reforming or oxygen-fed, autothermal reforming, especially steam or autothermal reforming of methane-containing gas, is used. The depleted gas phase may also contain sulfur-containing compounds, alcohol and the like volatilized from the aqueous fermentation broth.

Product Recovery

The bioreactor may have added from time to time or continuously one or more streams of water, nutrients or adjuvants, and microorganisms. A portion of the aqueous fermentation broth is withdrawn from time to time or continuously from the bioreactor for product recovery. Usually, the withdrawal is made at a point at the upper portion of the aqueous fermentation broth in the bioreactor. Product recovery can consist of known equipment arrangements for removal of residual cell material, separation and recovery of liquid products from the fermentation liquid, return of recovered fermentation liquid and purging of waste streams and materials. Suitable equipment arrangements can include filters, centrifuges, cyclones, distillation columns, membrane systems and other separation equipment. U.S. Pat. No. 8,211,679 shows an arrangement for a product recovery bioreactor that recovers an ethanol product from a bioreactor.

It is claimed:

1. A continuous process for the anaerobic bioconversion of a gas substrate comprising carbon monoxide, hydrogen and carbon dioxide in an aqueous menstruum containing microorganisms suitable for converting said substrate to oxygenated organic compound, comprising: continuously contacting said gas substrate with said aqueous menstruum under acidic and anaerobic fermentation conditions to bioconvert gas substrate to oxygenated organic compound and provide an oxygenated organic compound-containing menstruum and a depleted gas phase; continuously withdrawing the depleted gas phase from said aqueous menstruum; continuously or intermittently withdrawing a portion of said menstruum for recovery of said oxygenated organic compound, said withdrawal being sufficient to maintain the oxygenated organic compound in said menstruum below a concentration that unduly adversely affects the microorganisms, wherein during the contacting the aqueous menstruum contains undissolved calcium sulfite.

2. The process of claim 1 wherein the pH of the aqueous menstruum is between about 4 and 6.0.

3. The process of claim 2 wherein the pH of the aqueous menstruum is between about 4.5 and 5.5.

4. The process of claim 2 wherein at least a portion of the undissolved calcium sulfite is an in situ precipitate occurring in the aqueous menstruum.

5. The process of claim 2 wherein at least a portion of the undissolved calcium sulfite is supplied to the aqueous menstruum by adding solid calcium sulfite to the aqueous menstruum.

6. The process of claim 5 wherein at least about 50 mass percent of the solid calcium sulfite added to the aqueous menstruum have a maximum particle size dimension of between about 1 and 100 microns.

7. The process of claim 5 wherein the addition of calcium sulfite is continuous.

8. The process of claim 5 wherein the addition of calcium sulfite is intermittent.

9. The process of claim 1 wherein the undissolved calcium sulfite is substantially uniformly distributed in the aqueous menstruum.

10. A continuous process for the anaerobic bioconversion of a gas substrate comprising carbon monoxide, hydrogen and carbon dioxide in an aqueous menstruum containing microorganisms suitable for converting said substrate to oxygenated organic compound, comprising: continuously contacting said gas substrate with said aqueous menstruum under acidic anaerobic fermentation conditions to bioconvert gas substrate to oxygenated organic compound and provide an oxygenated organic compound-containing menstruum and a depleted gas phase; continuously withdrawing the depleted gas phase from said aqueous menstruum;
continuously or intermittently withdrawing a portion of said menstruum for recovery of said oxygenated organic compound, said withdrawal being sufficient to maintain the oxygenated organic compound in said menstruum below a concentration that unduly adversely affects the microorganisms, wherein during the contacting undissolved calcium sulfite is provided to the aqueous menstruum in an amount sufficient to maintain undissolved calcium sulfite in the aqueous menstruum.

11. The process of claim 10 wherein the addition of calcium sulfite is continuous.

12. The process of claim 10 wherein the addition of calcium sulfite is intermittent.

13. The process of claim 10 wherein the calcium sulfite is added as an aqueous slurry.

14. The process of claim 10 wherein the calcium sulfite added comprises calcium sulfite solids having a maximum particle size dimension of between about 1 and 100 microns.

15. The process of claim 10 wherein the oxygenated organic compound comprises at least one of ethanol, propanol and butanol.
